(19) United States
(12) Patent Application Publication
(10) Pub. No.: US 2019/0313918 A1
(43) Pub. Date: Oct. 17, 2019

(54) ARTERIAL PULSE SIGNAL MEASUREMENT DEVICE AND PRESSURE SENSOR

(71) Applicant: SHENZHEN NEW DEGREE TECHNOLOGY CO., LTD., Shenzhen, Guangdong (CN)

(72) Inventors: Hao LI, Shenzhen, Guangdong (CN); Fei CHEN, Shenzhen, Guangdong (CN)

(21) Appl. No.: 16/305,555

(22) PCT Filed: May 31, 2016
(86) PCT No.: PCT/CN2016/084186

§ 371 Date: Nov. 29, 2018

(51) Int. Cl. A61B 5/024; A61B 5/021; A61B 5/00; G01L 9/02

(52) U.S. Cl. CPC A61B 5/024; A61B 5/021; A61B 2562/04; G01L 9/02; A61B 2562/0247; A61B 5/6801

(57) ABSTRACT

An arterial pulse signal measurement device includes a pressure sensor and a measuring apparatus. When the pressure sensor is attached to the skin, stratum corneum or other superficial organs of a human body or animal body, and a constant voltage is applied to first voltage input terminal (V+) and a second voltage input terminal (V−) of the pressure sensor, the pressure sensor will output a corresponding voltage signal to the measuring apparatus according to the arterial pulse of the human body or animal body. The measuring apparatus will acquire the arterial pulse signal of the human body or animal body according to the voltage signal.

### TECHNICAL FIELD

The present disclosure relates to a field of medical devices, particularly to an arterial pulse signal measurement device and a pressure sensor thereof.

### BACKGROUND

The arterial pulse signal refers to a pulse signal caused by an artery pumping blood with a heart, which is resulted from changes in the pressure of blood pulse on the vessel wall and generated by the blood pumping activity of the heart. This signal is directly related with physiological indexes such as a heart rate, a blood pressure, a blood fat, a hemodynamic change and the like.

At present the methods for measuring the arterial pulse signal includes an invasive measurement and a non-invasive measurement. The non-invasive measurement usually involves changing the blood flow by compressing the artery, and then measuring the arterial pulse signal according to the sound or vibration during the change of the blood flow. This kind of method relieves patients' pain greatly with its inherent noninvasive surgery, and is adopted widely for its advantages such as low dependence on equipment and professionalism of medical staffs and the like.

However, when the arterial pulse signal is measured by using the traditional non-invasive measurement, an additional pressure device (mostly an inflated and pressurized wrist band) is required to be provided for all traditional non-invasive measurement devices, which results in more components of the measurement devices, inconvenience to carry with, high cost and restriction for service environment.

### SUMMARY

The purpose of the present disclosure is to provide an arterial pulse signal measurement device and a pressure sensor thereof, aiming to solve the problems that the traditional non-invasive measurement devices, for which an additional pressure device is required to be provided, result in more components of the measurement devices, inconvenience to carry with, high cost and restriction for service environment.

The present disclosure is implemented as follows: a pressure sensor of an arterial pulse signal measurement device, which is connected to a measuring apparatus in the arterial pulse signal measurement device, and the pressure sensor includes a substrate and a pressure sensing detection circuit arranged on the substrate;

the pressure sensing detection circuit includes: a first resistor, a second resistor, a third resistor and a fourth resistor, wherein at least one of the first resistor, the second resistor, the third resistor and the fourth resistor is a strain sensing resistor;

the first resistor, the third resistor, the second resistor and the fourth resistor are electrically connected sequentially to form a full bridge circuit, the common terminal of the first resistor and the fourth resistor is a first voltage input terminal of the pressure sensor, the common terminal of the second resistor and the third resistor is a second voltage input terminal of the pressure sensor, the common terminal of the first resistor and the third resistor is a voltage output terminal of pressure sensor and connects to the measuring apparatus, and the common terminal of the second resistor and the fourth resistor is grounded;

when the pressure sensor is attached to the skin, the stratum corneum or other superficial organs of a human body or animal body through the substrate, and the first voltage input terminal and the second voltage input terminal of the pressure sensor are applied with a constant voltage, the pressure sensor will output corresponding voltage signal, according to the arterial pulse of the human body or animal body, to the measuring apparatus, and the measuring apparatus will acquire the arterial pulse signal of the human body or animal body according to said voltage signal.

The present disclosure further provides an arterial pulse signal measurement device, which includes a measuring apparatus and the above pressure sensor.

The present disclosure adopts the arterial pulse signal measurement device including the pressure sensor and the measuring apparatus, when the pressure sensor is attached to the skin, the stratum corneum or other superficial organs of the human body or animal body, and the first voltage input terminal and the second voltage input terminal of the pressure sensor are applied with a constant voltage, the pressure sensor will output corresponding voltage signal to the measuring apparatus according to the arterial pulse of the human body or animal body, and the measuring apparatus will acquire the arterial pulse signal of the human body or animal body according to said voltage signal. The arterial pulse signal measurement device provided by the present disclosure has a simple structure, and can implement the measurement of the pulse signal for the human body or animal body without the requirement of an additional pressurized device, which is cost-saving, convenient to carry, and is not restricted by the service environment.

### BRIEF DESCRIPTION OF THE DRAWINGS

FIG. 1 is a structural schematic view of the arterial pulse signal measurement device provided by an embodiment of the present disclosure;

FIG. 2 is a schematic view of a cross section of the pressure sensor provided by an embodiment of the present disclosure;

FIG. 3 is a schematic view of a circuit structure of the pressure sensing detection circuit in the pressure sensor provided by an embodiment of the present disclosure;

FIG. 4(a) and FIG. 4(B) are respectively structural schematic diagrams of the front and back sides of the pressure sensor provided by an embodiment of the present disclosure;

FIG. 5 is a schematic view of a cross section of the pressure sensor provided by another embodiment of the present disclosure;

FIG. 6 is a schematic view of the pressure sensor provided by an embodiment of the present disclosure, which is fitted to a part of the human body to be measured by a double-surfaced adhesive;

FIG. 7 is a schematic view of the pressure sensor provided by an embodiment of the present disclosure, which is fitted to a part of the human body to be measured by a medical proof fabric.

### DETAILED EMBODIMENTS

In order to illustrate the purpose, the technical solutions and advantages of the present disclosure more clearly, the present disclosure will be further described below with reference to the drawings and embodiments. It should be understood that, specific embodiments described herein are merely to explain the present disclosure, and are not to limit the present disclosure.

The embodiments of the present disclosure provide an arterial pulse signal measurement device and a pressure sensor of the device.

FIG. 1 is a structural schematic view of the arterial pulse signal measurement device provided by an embodiment of the present disclosure, for convenience of description, only parts related to the present disclosure are shown, which are described in detail as follows:

As shown in FIG. 1, the arterial pulse signal measurement device includes a pressure sensor and a measuring apparatus 2. Wherein, the pressure sensor 1 is used to attach to the skin, the stratum corneum or other parts with micro-deformation characteristics of the human body or animal body, and output corresponding voltage signal to the measuring apparatus 2 according to the arterial pulse of the human body or animal body, the measuring apparatus 2 is used to acquire the arterial pulse signal of the human body or animal body according to said voltage signal.

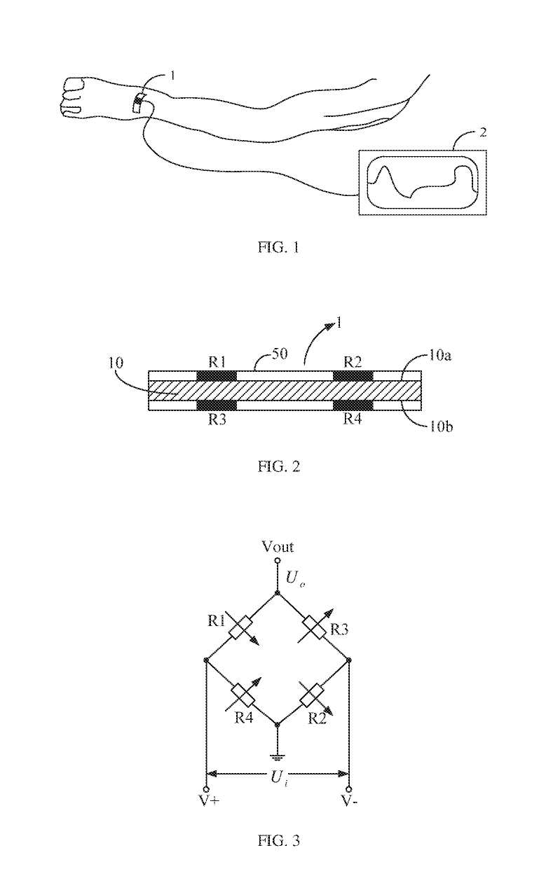

FIG. 2 is a cross section view of the pressure sensor provided by an embodiment of the present disclosure, for convenience of description, only parts related to the present disclosure are shown, which are described in detail as follows:

As shown in FIG. 2, the pressure sensor 1 includes a substrate 10 and a pressure sensing detection circuit arranged on the substrate 10, wherein, the pressure sensing detection circuit is used to detect the arterial pulse signal of the human body or animal body, and the pressure sensing detection circuit includes: a first resistor R1, a second resistor R2, a third resistor R3 and a fourth resistor R4.

At least one resistor of the first resistor R1, the second resistor R2, the third resistor R3 and the fourth resistor R4 is strain sensing resistor.

FIG. 3 is a circuit structure view of the pressure sensing detection circuit provided by an embodiment of the present disclosure, as shown in FIG. 3, the first resistor R1, the third resistor R3, the second resistor R2 and the fourth resistor R4 are electrically connected sequentially to form a full bridge circuit, the common terminal of the first resistor R1 and the fourth resistor R4 is the first voltage input terminal V+ of pressure sensor 1, the common terminal of the second resistor R2 and the third resistor R3 is the second voltage input terminal V− of pressure sensor 1, the common terminal of the first resistor R1 and the third resistor R3 is the voltage output terminal Vout of pressure sensor 1, and the common terminal of the third resistor R4 and the second resistor R2 is grounded.

In the present embodiment, when the strain sensing resistors are deformed due to external force, the value of the resistor will change correspondingly, thereby, when a constant voltage is applied between the first voltage input terminal and the second voltage input terminal of the pressure sensor 1, the voltage of the voltage output terminal of the pressure sensor 1 changes with the resistance change of the strain resistor.

In practical applications, when the pressure sensor 1 is applied to the skin, the stratum corneum or other parts with micro-deformation characteristics of the human body or animal body, and the first voltage input terminal and the second voltage input terminal of the pressure sensor 1 are applied with a constant voltage, the voltage output terminal of the pressure sensor 1 will output corresponding voltage signal to the measuring apparatus 2 according to the arterial pulse of the human body, and the measuring apparatus 2 will analyze and process the voltage signal to acquire the arterial pulse signal of the human body or animal body.

In the embodiment of the present disclosure, the pressure sensor 1 may transmit the voltage signal to the measuring apparatus 2 through a wired or wireless mode (the wired transmission mode in FIG. 1 is taken as an example), wherein, the wireless transmission mode includes, but is not limited to transmission modes such as Bluetooth, WIFI, ZigBee and the like.

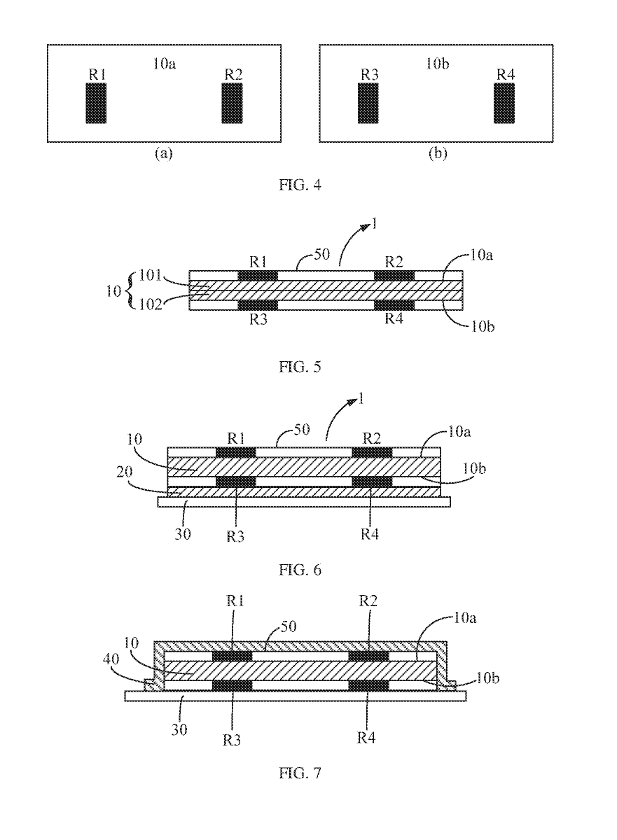

FIG. 4(a) and FIG. 4(B) are the respective structural schematic diagrams of the front and back sides of the pressure sensor provided by an embodiment of the present disclosure, for convenience of description, only parts related with the present disclosure are shown, which are described in detail as follows:

As shown in FIG. 2 and FIG. 4, the substrate 10 includes a front side 10a and a back side 10b, each of the front side 10a and the back side 10b may be used as one surface of the substrate 10 attaching to the skin, the stratum corneum or other parts with micro-deformation characteristics of the human body or animal body. It should be noted that, the front side 10a and the back side 10b of the substrate 10 have no substantial difference, merely to show that the two surfaces of the substrate are oppositely arranged.

As an embodiment of the present disclosure, one resistor of the first resistor R1, the second resistor R2, the third resistor R2 and the fourth resistor R4 is arranged on the front side 10a of the substrate, and other three resistors are arranged on the back side 10b of the substrate.

As another embodiment of the present disclosure, one resistor of the first resistor R1, the second resistor R2, the third resistor R2 and the fourth resistor R4 is arranged on the back side 10b of the substrate, and other three resistors are arranged on the front side 10a of the substrate.

As another embodiment of the present disclosure, two resistors of the first resistor R1, the second resistor R2, the third resistor R2 and the fourth resistor R4 are arranged on the front side 10a of the substrate, and other two resistors are arranged on the back side 10b of the substrate.

As another embodiment of the present disclosure, the first resistor R1, the second resistor R2, the third resistor R2 and the fourth resistor R4 are arranged on the front side 10a of the substrate.

As another embodiment of the present disclosure, the first resistor R1, the second resistor R2, the third resistor R2 and the fourth resistor R4 are arranged on the back side 10b of the substrate.

In embodiments of the present disclosure, specifically, it should be determined according to actual conditions which resistors are arranged on the front side 10a of the substrate 10 and which resistors are arranged on the back side 10b of the substrate 10, which are not listed herein one by one.

As an embodiment of the present disclosure, all resistors are non-strain sensing resistors in the first resistor R1, the second resistor R2, the third resistor R3 and the fourth resistor R4 except strain sensing resistors.

As an embodiment of the present disclosure, one resistor of the first resistor R1, the second resistor R2, the third resistor R3 and the fourth resistor R4 is strain sensing resistor, and another three resistors are non-strain sensing resistors.

As another embodiment of the present disclosure, two resistors of the first resistor R1, the second resistor R2, the third resistor R3 and the fourth resistor R4 are strain sensing resistors, and another two resistors are non-strain sensing resistors.

As another embodiment of the present disclosure, three resistors of the first resistor R1, the second resistor R2, the third resistor R3 and the fourth resistor R4 are strain sensing resistors, and another one resistor is non-strain sensing resistor.

As another embodiment of the present disclosure, the first resistor R1, the second resistor R2, the third resistor R3 and the fourth resistor R4 are strain sensing resistors.

As an embodiment of the present disclosure, when the first resistor R1, the second resistor R2, the third resistor R3 and the fourth resistor R4 include a plurality of strain sensing resistors, the properties of the plurality of strain sensing resistors are the same.

As another embodiment of the present disclosure, the substrate 10 may adopt a flexible printed circuit board.

As another embodiment of the present disclosure, shown as FIG. 5, the substrate 10 includes a first flexible printed circuit board 101 and a second flexible printed circuit board 102, the first flexible printed circuit board 101 and the second flexible printed circuit board 102 fit closely to form the substrate 10, and a non-attached surface of the first flexible printed circuit board 101 and a non-attached surface of the second flexible printed circuit board 102 form the front side 10a and the back side 10b of the substrate 10 respectively. Wherein, a thickness of the flexible printed circuit board is about 0.08 mm-3 mm.

As an embodiment of the present disclosure, the substrate 10 is provided with a first voltage input port, a second voltage input port and a voltage output port corresponding to the first voltage input terminal, the second voltage input terminal and the voltage output terminal of the pressure sensor 1, in practical measurement, the pressure sensor 1 is attached to the part of the human body or the animal body to be measured, and the pressure sensor 1 is connected to the measuring apparatus 2 through the voltage output port of the substrate 10, then an input voltage is applied between the first voltage input port and the second voltage input port to implement measurement of the arterial pulse signal of the human body or animal body.

As an embodiment of the present disclosure, the pressure sensor 1 further includes an outermost layer attached to the pressure sensor 1, and a protective layer 50 protecting the pressure sensor 1.

As an embodiment of the present disclosure, the measuring apparatus includes a signal processing unit and a display unit.

The signal processing unit is used to analyze and process the voltage signal, such as to acquire the arterial pulse signal of the human body or animal body, and to acquire various physiological parameters of the human body or animal body (including, but not limited to heart rate, blood pressure, blood lipids, etc.) according to the arterial pulse signal of the human body or animal body; the display unit is connected to the signal processing unit, and is used to display the various physiological parameters of the human body or animal body. Wherein, the signal processing unit may specifically be a single chip microcomputer, an ARM (Advanced RISC Machines) processor or other programmable control device with data logic processing capability; the display unit may specifically be an LCD (Liquid Crystal Display) display, an LED (Light-emitting Diode) display or other types of display screens.

In embodiments of the present disclosure, the measuring apparatus 2 includes, but is not limited to terminals capable of signal analysis and result display such as a computer, a telephone, an electronic watch, a household measuring instrument and the like.

In practical applications, when measuring the arterial pulse signal of the human body or animal body, shown as FIG. 6 and FIG. 7, the pressure sensor 1 may either be attached to the part 30 of the human body or animal body to be measured through a double-surfaced adhesive 20; or the pressure sensor 1 may be first attached to a medical proof fabric 40, then the medical proof fabric 40 fitted with the pressure sensor 1 is fitted to the part 30 of the human body or animal body to be measured.

The measurement of the arterial pulse signal provided by above embodiments will be further described below by reference to the working principles:

When measuring the arterial pulse signal of the human body or animal body, the voltage output terminal of the pressure sensor 1 is first connected to the measuring apparatus 2, then the pressure sensor 1 is attached to the part of the human body or animal body to be measured, and the skin, the stratum corneum or other superficial organs of the part of the human body or animal body to be measured will deform with the pulsation of the arteries. When the skin, the stratum corneum or other superficial organs of the part of the human body or animal body to be measured are deformed, the strain sensing resistors in the pressure sensor 1 attached to the skin, the stratum corneum or other superficial organs of the human body or animal body generates different degree of deformation, which results in change of resistance of the strain sensing resistors. when the first voltage input terminal and the second voltage input terminal of the pressure sensor 1 is applied with a constant input voltage, the change of the resistance of the strain sensing resistors causes the output voltage of the voltage output terminal of the pressure sensor 1 to change, and the measuring apparatus 2 analyzes and processes the voltage signal of the voltage output terminal of the pressure sensor 1 to acquire the arterial pulse signal of the human body or animal body, and to obtain the various physiological parameters of the human body or animal body (including, but not limited to heart rate, blood pressure, blood lipids, etc.) according to the arterial pulse signal of the human body or animal body, meanwhile, the measuring apparatus 2 displays the various physiological parameters of the human body or animal body.

In practical applications, the pressure sensor 1 may be directly attached to the wrist artery, the fingernail cover or other skin, stratum corneum or other superficial organs, with obvious arterial pulse signal, of the human body to measure the heart rate of the human body; the pressure sensor 1 may also be integrated into smart wearable devices such as sports bracelets, electronic watches, heart rate belts, etc., such as to measure the physiological change characteristics such as the heart rate and the blood pressure of the human body through combining the arterial pulse signal measured by the pressure sensor 1 with the bioelectrical and photoelectric sensor data of the human body measured by the smart wearable devices; the two sets of pressure sensors 1 may also be respectively attached to a proximal end of the human limbs near the heart and a distal end of the human limbs away from the heart, such that to measure the blood pressure of the human body by calculating the phase difference of the arterial pulse signals collected by the two sets of pressure sensors 1, and measure the physiological characteristics such as arterial occlusion and arterial wall health and the like according to the lantency of the phase difference; or several sets of pressure sensors 1 are respectively attached to the part of the human body such as the abdominal cavity, chest cavity, pelvic cavity and the like, and the deformation waveforms of different parts of the body cavity generated with respiratory change are collected, so as to measure the pressure value of the body cavity according to the phase difference and signal change rate of the deformation waveforms in different parts of the cavity. It should be noted that, the application scenarios of the arterial pulse signal measurement device provided by embodiments of the present disclosure include but is not limited to above application scenarios.

The embodiments of the present disclosure adopt the arterial pulse signal measurement device including the pressure sensor and the measuring apparatus, when the pressure sensor is attached to the skin, the stratum corneum or other superficial organs of the human body or animal body, and the first voltage input terminal and the second voltage input terminal of the pressure sensor are applied with a constant voltage, the pressure sensor will output corresponding voltage signal to the measuring apparatus according to the arterial pulse of the human body or animal body, and the measuring apparatus will acquire the arterial pulse signal of the human body or animal body according to the voltage signal. The arterial pulse signal measurement device. provided by embodiments of the present disclosure has a simple structure, and can implement the measurement of the pulse signal for the human body or animal body without the requirement of an additional pressurized device, which is cost-saving, convenient to carry, and is not restricted by the service environment.

The above description is only preferred embodiments of the present disclosure, and is not intended to limit the present disclosure. Any modifications, equivalent substitutions and improvements made within the spirit and principles of the present disclosure should be included in the scope of the present disclosure.

1. A pressure sensor of an arterial pulse signal measurement device, configured to be connected to a measuring apparatus in the arterial pulse signal measurement device, the pressure sensor comprising:
a substrate;
a pressure sensing detection circuit arranged on the substrate;
the pressure sensing detection circuit comprising a first resistor, a second resistor, a third resistor and a fourth resistor, wherein at least one of the first resistor, the second resistor, the third resistor and the fourth resistor comprises a strain sensing resistor;
wherein the first resistor, the third resistor, the second resistor and the fourth resistor are electrically connected sequentially to form a full bridge circuit, a common terminal of the first resistor and the fourth resistor providing a first voltage input terminal of the pressure sensor, a common terminal of the second resistor and the third resistor providing a second voltage input terminal of the pressure sensor, a common terminal of the first resistor and the third resistor providing a voltage output terminal of the pressure sensor and being configured to connect with the measuring apparatus, and a common terminal of the second resistor and the fourth resistor being configured to be grounded;
wherein when the pressure sensor is applied to the skin, stratum corneum or another superficial organ of a human body or animal body, and the first voltage input terminal and the second voltage input terminal of the pressure sensor are applied with a constant voltage, the pressure sensor will output a corresponding voltage signal to the measuring apparatus according to an arterial pulse of the human body or animal body, and the measuring apparatus will acquire an arterial pulse signal of the human body or animal body according to said voltage signal.

2. The pressure sensor of claim 1, wherein the substrate comprises a front side and a back side, and each of the front side and the back side acts as one side of the substrate attachable to the skin, stratum corneum or other superficial organs of the human body or animal body.

3. The pressure sensor of claim 2, wherein one of the first resistor, the second resistor, the third resistor and the fourth resistor is arranged on the front side of the substrate, and the other three resistors are arranged on the back side of the substrate.

4. The pressure sensor of claim 2, wherein one resistor of the first resistor, the second resistor, the third resistor and the fourth resistor is arranged on the back side of the substrate, and the other three resistors are arranged on the front side of the substrate.

5. The pressure sensor of claim 2, wherein two resistors of the first resistor, the second resistor, the third resistor and the fourth resistor are arranged on the front side of the substrate, and the other two resistors are arranged on the back side of the substrate.

6. The pressure sensor of claim 2, wherein the first resistor, the second resistor, the third resistor and the fourth resistor are all arranged on the front side of the substrate.

7. The pressure sensor of claim 2, wherein the first resistor, the second resistor, the third resistor and the fourth resistor are all arranged on the back side of the substrate.

8. The pressure sensor of claim 1, wherein the substrate is a flexible printed circuit board.

9. The pressure sensor of claim 1, wherein the substrate comprises a first flexible printed circuit board and a second flexible printed circuit board, the first flexible printed circuit board and the second flexible printed circuit board are arrange closely to each other forming the substrate, and a non-attached surface of the first flexible printed circuit board and a non-attached surface of the second flexible printed circuit board form the front side and the back side of the substrate respectively.

10. The pressure sensor of claim 1, wherein each of the first resistor, the second resistor, the third resistor and the fourth resistor is non-strain sensing resistor except being the strain sensing resistor.

11. An arterial pulse signal measurement device, comprising a measuring apparatus, wherein the arterial pulse signal measurement device further comprising the pressure sensor according to claim 1.

12. The arterial pulse signal measurement device of claim 11, wherein the measuring apparatus comprises a signal processing unit and a display unit;
wherein the signal processing unit is configured to analyze and process the voltage signal to acquire an arterial pulse signal of the human body or animal body, and to acquire physiological parameter of the human body or animal body according to the arterial pulse signal of the human body or animal body; and
wherein the display unit is configured to be connected to the signal processing unit, and to display the physiological parameter of the human body or animal body.

13. An arterial pulse signal measurement device, comprising a measuring apparatus, wherein the arterial pulse signal measurement device further comprising the pressure sensor according to claim 2.

14. An arterial pulse signal measurement device, comprising a measuring apparatus, wherein the arterial pulse signal measurement device further comprising the pressure sensor according to claim 3.

15. An arterial pulse signal measurement device, comprising a measuring apparatus, wherein the arterial pulse signal measurement device further comprising the pressure sensor according to claim 4.

16. An arterial pulse signal measurement device, comprising a measuring apparatus, wherein the arterial pulse signal measurement device further comprising the pressure sensor according to claim 5.

17. An arterial pulse signal measurement device, comprising a measuring apparatus, wherein the arterial pulse signal measurement device further comprising the pressure sensor according to claim 6.

18. An arterial pulse signal measurement device, comprising a measuring apparatus, wherein the arterial pulse signal measurement device further comprising the pressure sensor according to claim 7.

19. An arterial pulse signal measurement device, comprising a measuring apparatus, wherein the arterial pulse signal measurement device further comprising the pressure sensor according to claim 8.

20. A method for acquiring an arterial pulse signal of a human or animal body, comprising:
applying a pressure sensor to skin, stratum corneum or another superficial organ of a human or animal body, the pressure sensor comprising:
a substrate; and
a pressure sensing detection circuit arranged on the substrate, the pressure sensing detection circuit comprising a first resistor, a second resistor, a third resistor and a fourth resistor, wherein at least one of the first resistor, the second resistor, the third resistor and the fourth resistor comprises a strain sensing resistor, wherein the first resistor, the third resistor, the second resistor and the fourth resistor are electrically connected sequentially to form a full bridge circuit, a common terminal of the first resistor and the fourth resistor providing a first voltage input terminal, a common terminal of the second resistor and the third resistor providing a second voltage input terminal, a common terminal of the first resistor and the third resistor providing a voltage output terminal, and a common terminal of the second resistor and the fourth resistor being grounded;
applying a constant voltage to the first voltage input terminal and the second voltage input terminal;
producing, with the pressure sensor at the pressure sensor voltage output terminal, a voltage signal according to an the arterial pulse of the human or animal body, and
acquiring an arterial pulse signal of the human or animal body according to said voltage signal.
